(19) United States
(12) Patent Application Publication
(10) Pub. No.: US 2020/0359620 A1
(43) Pub. Date: Nov. 19, 2020

(54) CLEANING AND DISINFECTING WIPE WITH INDICATION OF PROPER USE

(71) Applicants: Karla Abela, Pearland, TX (US); Anthony Bentley, Seabrook, TX (US); Tammy Hoy, League City, TX (US); Heather Mckelvy, Menlo Park, CA (US); Scott Naples, Hudson, OH (US); Ingrid Parry, Carmichael, CA (US); Jeffrey Hovis, Boston, MA (US)

(72) Inventors: Karla Abela, Pearland, TX (US); Anthony Bentley, Seabrook, TX (US); Tammy Hoy, League City, TX (US); Heather Mckelvy, Menlo Park, CA (US); Scott Naples, Hudson, OH (US); Ingrid Parry, Carmichael, CA (US); Jeffrey Hovis, Boston, MA (US)

(21) Appl. No.: 16/304,072

(22) PCT Filed: May 23, 2017
(86) PCT No.: PCT/US2017/034037

§ 371 Date: Nov. 21, 2018

Related U.S. Application Data
(60) Provisional application No. 62/339,993, filed on May 23, 2016.

(51) Int. Cl. A01N 25/34; A47L 13/17; A61F 13/84

(52) U.S. Cl. CPC A01N 25/34; A61F 2013/15048; A61F 13/8405; A47L 13/17

(57) ABSTRACT

A disposable wipe comprising a main body including a first surface and a second surface. The first surface is comprised of one or more layers of a cleaning material and at least one of a chemical or a chemical solution. The second surface is comprised of a plurality of layers, at least one of the layers comprising an indication system for indicating proper use of the disposable wipe.

### CROSS-REFERENCE TO RELATED APPLICATION

This application claims the benefit of U.S. Provisional Application No. 62/339,993 filed May 23, 2016, which is hereby incorporated by reference in its entirety.

### FIELD OF THE INVENTION

The present invention relates to a cleaning and disinfecting wipe with two functional surfaces including a cleaning surface and an indication surface. The indication surface includes an embedded layer of one or more indicators for indicating proper use of the wipe. The indicators provide an indication of proper scrubbing and cleaning time and optionally, proper waiting or drying time after scrubbing.

### BACKGROUND OF THE INVENTION

Cleaning and disinfecting wipes are widely used by medical personnel. The wipes are designed to clean and disinfect patient skin sites, medical equipment and other surfaces. To perform properly, wipes should be used for an adequate amount of time to be effective in removing unwanted materials, such as body fluids, from surfaces. Additionally, wipes should be used with an adequate amount of pressure to ensure the removal of such unwanted materials.

Thus, it is desirable to provide a cleaning and disinfecting wipe that provides an indication that the proper amount of time and applied pressure has been used for cleaning and/or disinfecting purposes. Moreover, it is desirable to provide a cleaning and disinfecting wipe that provides an indication that a surface is clean and/or dry.

### SUMMARY OF THE INVENTION

According to one embodiment, a disposable wipe comprises a main body including a first surface and a second surface. The first surface is comprised of one or more layers of a cleaning material and at least one of a chemical or a chemical solution. The second surface is comprised of a plurality of layers. At least one of the layers comprises an indication system for indicating a proper use of the disposable wipe.

According to a further embodiment, a method of making a disposable wipe comprises providing a main body including a first surface and a second surface, wherein the first surface is comprised of one or more layers of a cleaning material and one or more of a chemical or a chemical solution, and the second surface is comprised of a plurality of layers. The method also comprises embedding at least one of the plurality of layers with an indication system for indicating proper use of the disposable wipe.

### BRIEF DESCRIPTION OF THE DRAWINGS

The foregoing and other advantages of the invention will become apparent upon reading the following detailed description and upon reference to the drawings.

FIG. 1 is a perspective view of a disposable wipe having a first surface and a second surface.

FIG. 2 is a perspective view of the second surface of FIG. 1.

FIG. 3 is a perspective view of the disposable wipe of FIG. 1 with an indication that a proper scrubbing and cleaning time has occurred.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

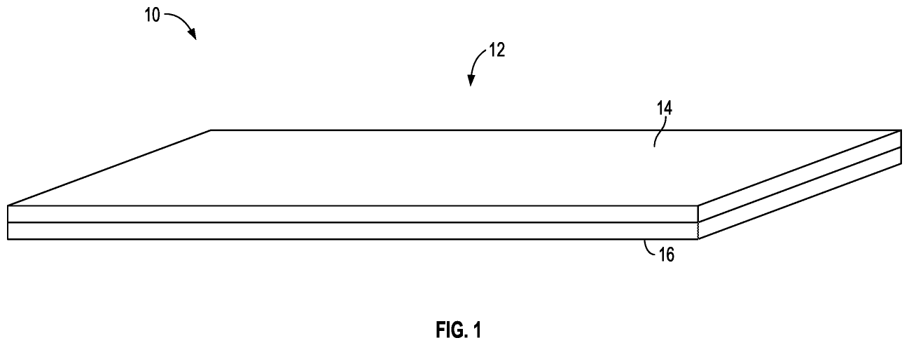

FIG. 1 illustrates a disposable wipe 10 according to one embodiment of the present invention. The disposable wipe 10 comprises a main body 12 having a first surface 14 and a second surface 16. The first surface 14 is a cleaning and/or disinfecting surface. The first surface is comprised of one or more layers of cleaning materials. The cleaning materials may include a woven fabric, a non-woven fabric, or a combination thereof.

The first surface 14 is also comprised of a cleaning and/or disinfecting chemical and/or a chemical solution. The cleaning and/or disinfecting chemical and/or chemical solution may include isopropyl alcohol, ethyl alcohol, chlorhexidine gluconate solution, providone-iodine solution, hydrogen peroxide solution, sodium hypochlorite solution, a detergent solution, or a combination thereof. In some embodiments, the amount of cleaning and/or disinfecting chemical and/or chemical solution should be enough to saturate the first surface 14, however, lesser amounts may also be used. The first surface 14 may also be textured to aid in the cleaning and/or disinfecting effectiveness. The textures may include, for example, a raised or rough texture that provides a different touch, feel or consistency to the first surface 14 and which aids in the cleaning and/or disinfecting process.

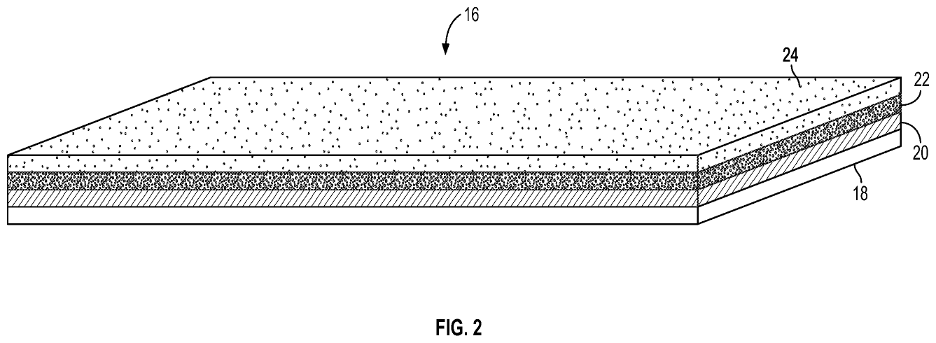

The second surface 16 is an indicating surface. The second surface may be comprised of a plurality of layers, as shown in FIG. 2. The layers may include one or more of the following: a carrier layer 18, a barrier layer 20, an indicator layer 22, and a surface protection layer 24. The carrier layer 18 is adjacent the one or more layers of the first surface 14. In some embodiments, the carrier layer 18 may be the back of the one or more layers of the first surface 14. In other embodiments, the carrier layer 18 and the barrier layer 20 may be combined into a single layer. The carrier layer 18 may be comprised of a woven fabric, a non-woven fabric, a paper or a polymer film.

The barrier layer 20 may be comprised of a barrier coating that is resistant to the movement or transport of chemicals or materials in the disposable wipe 10. In other embodiments, the barrier layer 20 may be comprised of a barrier polymer film that is also resistant to the movement or transport of chemicals and materials in the disposable wipe. At least one of the barrier layer 20 and the carrier layer 18 is opaque and provides a background color for the indicator layer 22. The indicator layer 22 is comprised of one or more microencapsulated indicator dyes and microencapsulated indicator dye developers in a binding chemistry, as discussed in more detail below.

The surface protection layer 24 may be comprised of a barrier coating that is resistant to the movement or transport of chemicals or materials in the disposable wipe 10. In other embodiments, the barrier layer 20 may be comprised of a barrier polymer film that is also resistant to the movement or transport of chemicals and materials in the disposable wipe. The surface protection layer 24 is transparent to allow the indicator layer 22 to be visible to the user of the disposable wipe 10.

The indicator layer 22 comprises an indication system for indicating proper use of the disposable wipe 10. Specifically, the indication system is designed to indicate the timing for the proper scrubbing or cleaning time for the disposable wipe 10. Optionally, the indication system can also indicate the timing for the proper waiting or drying time after scrubbing. The indication system may be comprised of one or more microencapsulated indicator dyes and/or one or more microencapsulated indicator dye developers as noted above. The dye may include a leuco dye and/or a leuco dye developer. A leuco dye is a dye which can switch between two chemical forms, one of which is colorless. The dye is FDA approved and is non-toxic. The microencapsulated indicator dye and the microencapsulated indicate dye developer system is designed to react after the proper amount of scrubbing time, based on the scrubbing force breaking the friable microcapsules. When that occurs, the dye is released, however, the dye does not contact the patient or device, such as a catheter hub or other medical device, due to the barrier coating or the barrier polymer film of the surface protection layer.

Once the microcapsules are ruptured, i.e, by scrubbing, a first indicator dye and indicator dye developer system reacts quickly to produce a first visible indication. A first dye reaction time takes place after a fixed amount of scrubbing time. In some embodiments, the first dye reaction time may be between 10 and 60 seconds. Optionally, a second indicator dye and indicator dye developer system is designed to react more slowly after rupture of the microcapsules to produce a second visible indication. A second dye reaction time takes place after a fixed amount of time after the end of the scrubbing time. In some embodiments, a second dye reaction time may be between 20 and 120 seconds.

In one embodiment, microencapsulation is produced by chemical microencapsulation. For the indicator dye and indicator dye developer system, the microencapsulation process may include interfacial polymerization, coacervation, in situ polymerization and liposomes. The microencapsulation process for the indicator dye and indicator dye developer system produces a friable shell with a large payload (>70%) and with a controllable rupture force. In some embodiments, the microencapsulation shell may be comprised of melamine formaldehyde, urea formaldehyde, isocyanate, gelatin, alginate, chitosan, natural polymers and synthetic polymers.

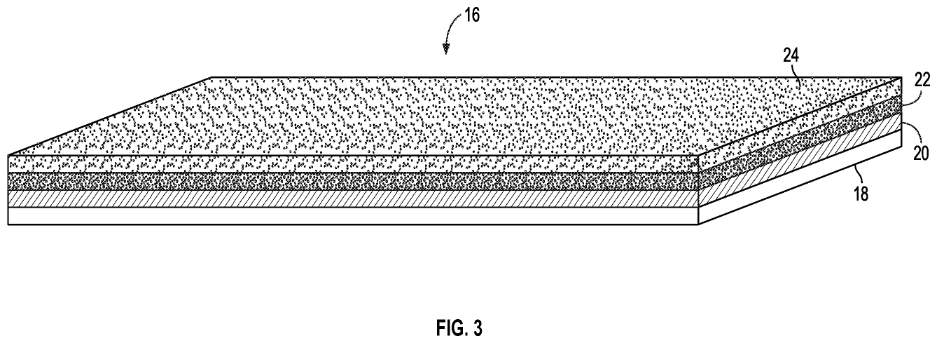

According to one embodiment of the present invention, the indication produced by the first indicator dye and indicator dye developer system may include a distinctive color, shape, pattern, symbol, or a combination of these elements. This first indication may cover all or a part of the second surface 16 so that the first indication, i.e., the color, shape, pattern or symbol, is able to be viewed by the user of the disposable wipe 10. The first indication is easily distinguished from the background color of the carrier layer 18 and the barrier layer 20. FIG. 3 provides an illustration of the second surface 16 of the disposable wipe after proper scrubbing and cleaning has occurred. In this example, the second surface is distinguishable (shown as a denser pattern as viewed through surface protection layer 24) when compared to FIG. 2 where proper scrubbing and cleaning has not yet occurred.

Optionally, in a further embodiment, a second indication may be produced by the second indicator dye and indicator dye developer system. The second indication may include a color, shape, pattern, symbol, or a combination of these elements which may cover all or a part of the second surface 16. The second indication is easily distinguished from the background color of the carrier layer 18 and the barrier layer 20 and the first indication. In another embodiment, the second indication may alternatively be created by reversing the first indication, i.e. by causing the first indication to disappear. For any of these embodiments, the colors associated with the carrier layer 18, the barrier layer 20, and the first and second indications are selected so that they are not easily confused with colors produced by bodily fluids. Such colors may include white, gray, black, purple, blue, dark green, bright orange, and others. The color, shape, pattern, symbol or combination of elements that are used for the first indication and the second indication may be determined based on a various factors such as a particular customer's requirements or preferences.

It is contemplated that the disposable wipe 10 of the present invention may include various shapes including round, oval, square, rectangular, or an irregular shape to aid in cleaning and/or disinfecting. Moreover, the form of the disposable wipe 10 may be planar, cup-shaped (with the cleaning surface on the inside), cone-shaped and cone (with a flat bottom)-shaped and fluted sides. It is contemplated that the indication system may include other colors, shapes, patterns or symbols in addition to those noted herein. Additionally, the disposable wipe 10 may be sterile or non-sterile.

According to one embodiment, the disposable wipe 10 may be made by providing a main body 12 having a first surface 14 and a second surface 16, wherein the first surface 14 is comprised of one or more layers of a cleaning material and one or more of a chemical or a chemical solution, and the second surface is comprised of a plurality of layers. The method further comprising embedding at least one of the layers of the second surface 16 with an indication system for indicating proper use of the disposable wipe.

In use, the disposable wipe 10 will change color (or show a shape, pattern or symbol) to indicate that an item, such as a catheter hub, is clean. The disposable wipe 10 may also change color to indicate that the hub is dry. The color changes indicate to clinicians and medical personnel that the correct amount of time and applied pressure to clean the hub has been used. Such an indication system can be used with alcohol wipes or CHG scrubs, and is application to cleaning skin sites, vial tops and other surfaces.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention, which is set forth in the following claims.

What is claimed is:

1. A disposable wipe comprising:
a main body including a first surface and a second surface,
wherein the first surface is comprised of one or more layers of a cleaning material and at least one of a chemical or a chemical solution, and
wherein the second surface is comprised of a plurality of layers, at least one of the layers comprising an indication system for indicating a proper use of the disposable wipe.

2. The disposable wipe of claim 1, wherein the one or more layers of the cleaning material comprises a woven fabric, a non-woven fabric, or a combination thereof.

3. The disposable wipe of claim 1, wherein chemical or chemical solution comprises at least one of isopropyl alcohol, ethyl alcohol, a chlorhexidine gluconate solution, a providone-iodine solution, a hydrogen peroxide solution, a sodium hypochlorite solution, a detergent solution, or a combination thereof.

4. The disposable wipe of claim 1, wherein the first surface is textured to aid cleaning and/or disinfecting effectiveness.

5. The disposable wipe of claim 1, wherein the indication system comprises one or more microencapsulated indicator dyes or microencapsulated indicator dye developers.

6. The disposable wipe of claim 1, wherein the plurality of layers of the second surface are comprised of at least one of a carrier layer, a barrier layer, an indicator layer and a surface protection layer.

7. The disposable wipe of claim 6, wherein the carrier layer is comprised of at least one of a woven fabric, a non-woven fabric, a paper and a polymer film.

8. The disposable wipe of claim 6, wherein at least one of the barrier layer and the surface protection layer is comprised of a barrier coating or a barrier polymer film that is resistant to movement of chemical materials.

9. The disposable wipe of claim 6, wherein the indicator layer comprises the indication system for indicating the timing for proper scrubbing or cleaning.

10. The disposable wipe of claim 9, wherein the indication system indicates the timing for proper scrubbing or cleaning via a distinctive color, shape, pattern, symbol, or a combination thereof.

11. A method of making a disposable wipe, the method comprising:
providing a main body including a first surface and a second surface, wherein the first surface is comprised of one or more layers of a cleaning material and one or more of a chemical or a chemical solution, and the second surface is comprised of a plurality of layers; and
embedding at least one of the plurality of layers with an indication system for indicating proper use of the disposable wipe.

12. The method of claim 11, wherein the indication system produces a color, shape, pattern or a combination thereof.

13. The method of claim 11, wherein the indication system indicates that a surface is clean.

14. The method of claim 11, wherein the indication system indicates that a surface is dry.

15. The method of claim 11, wherein the indication system indicates that a correct amount of time and pressure has been used.
